(19) United States
(12) Patent Application Publication
(10) Pub. No.: US 2015/0335878 A1
(43) Pub. Date: Nov. 26, 2015

(54) ALIGNMENT WRAP

(71) Applicants: Julie Jenks, Brentford (GB); Shaun Gayle, Chicago, IL (US)

(72) Inventors: Julie Jenks, Brentford (GB); Shaun Gayle, Chicago, IL (US)

(21) Appl. No.: 14/283,354

(22) Filed: May 21, 2014

(51) Int. Cl. A61N 1/05

(52) U.S. Cl. CPC A61N 1/0502

(57) ABSTRACT

Embodiments relate to an alignment device and method. The alignment device includes a wrap and an injection device for proper alignment and insertion of a metal device.

### FIELD OF THE INVENTION

The present invention is in the technical field of a medical device, specifically an alignment used with a body party to inset a wire or needle.

### BACKGROUND OF THE INVENTION

Overactive bladder (OAB) and fecal incontinence (FI) are well known, non-life threatening conditions. The major symptoms of OAB include urgency, frequent urination, nocturia (interruption of sleep to urinate) and urge incontinence (unintentionally urinating followed urge to continue). FI, also called faecal incontinence, bowel incontinence and anal incontinence, is a lack of control over defecation, leading to involuntary loss of bowel contents—including flatus, liquid stool elements and mucus, or solid feces.

There are generally two types or treatment. The first type of treatment, is a stimulator implant which is a small device surgically implanted under the skin. The stimulator implant transmits electrical pulses to the sacral nerves in the lower back which stimulate the nerves that that control the bladder.

The second type of treatment is Percutaneous tibial nerve stimulation (PTNS). PTNS requires a needle be inserted a couple of inches into the area proximate or above the ankle (medial malleolus). A grounding pad is attached to the patient's foot near the heel. The needle is in turned attached to a Transcutaneous electrical nerve stimulation (TENS) which creates electrical pulses used to treat OAB and/or FI. In general, patients choose not to insert needles into themselves and are leery that the needle would be inserted in the correct location. Therefore, the patient schedules an appointment with a medical practitioner, who inserts the needle and applies the stimulation.

It would therefore be desirable to have a device that properly aligns needle for insertion.

### SUMMARY OF THE INVENTION

Embodiments relate to an alignment device. The alignment includes a wrap; and an injection device for proper alignment and insertion of a metal device.

Other embodiments relate to an alignment device for use with a body for treatment of medical conditions such as OAB and/or FI. The alignment device includes a wrap having a first end and a second end for use in contact with a portion of the body; and a securement device for securing the first end to the second end. The alignment device further defines a tube having a first open end in contact with the portion of the human body, the alignment device providing proper alignment and insertion of a metal device.

Yet other embodiments relate to a method for inserting a metal device into a portion of a human body using an alignment device. The alignment device includes a wrap; and an injection device for proper alignment and insertion of the metal device. The method includes positioning the wrap on the portion of the human body; and slowly twisting and inserting the metal device.

The foregoing and other features and advantages of the invention will become further apparent from the following detailed description of the presently preferred embodiment, read in conjunction with the accompanying drawings. The drawings are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 illustrates an ankle of a patient suffering from OAB;

FIG. 2 illustrates a first view of the alignment device used with the ankle of FIG. 1 in accordance with one embodiment;

FIG. 3 illustrates a second view of the alignment device of FIG. 2;

FIG. 4 illustrates a first view of an alignment device in accordance with another embodiment used with an ankle similar to that of FIG. 1;

FIG. 5 illustrates a second view of the alignment device of FIG. 4;

FIG. 6 illustrates a first view of an alignment device in accordance with still another embodiment used with an ankle similar to that of FIG. 1;

FIG. 7 illustrates a second view of the alignment device of FIG. 6 used with the ankle of FIG. 1;

FIG. 8 illustrates a view of an alignment device in accordance with another embodiment used with an ankle similar to that of FIG. 1;

FIG. 9 illustrates a view of an alignment device in accordance with another embodiment used with an ankle similar to that of FIG. 1;

FIG. 10 illustrates a view of an alignment device in accordance with another embodiment used with an ankle similar to that of FIG. 1;

FIG. 11 illustrates a view of an alignment device in accordance with another embodiment used with an ankle similar to that of FIG. 1;

FIG. 12 illustrates a view of an alignment device in accordance with another embodiment used with an ankle similar to that of FIG. 1;

FIG. 13 illustrates an example of a TENS device coupled to a metal device or needle inserted a couple of inches into the patient's foot proximate or above the ankle; and

FIG. 14 illustrates a method for using the alignment device of FIGS. 2-12 with the patient's ankle of FIG. 1.

Throughout the various figures, like reference numbers refer to like elements.

### DETAILED DESCRIPTION OF PRESENTLY PREFERRED EMBODIMENTS

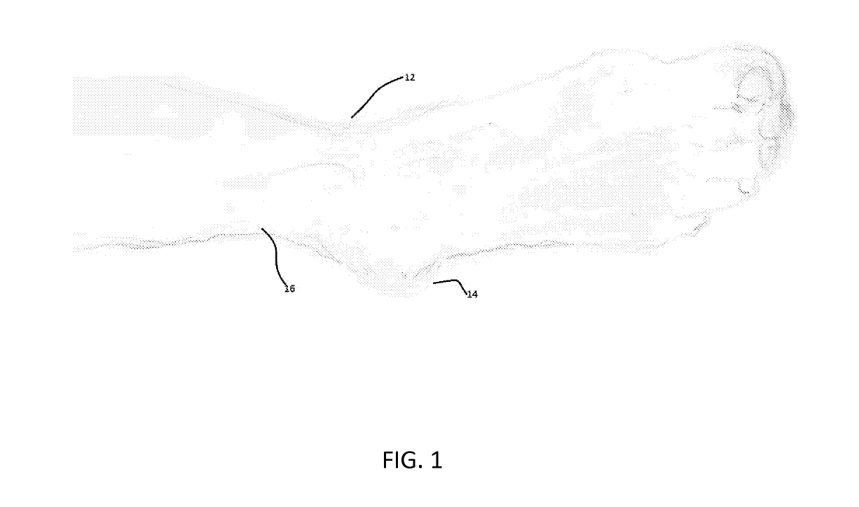

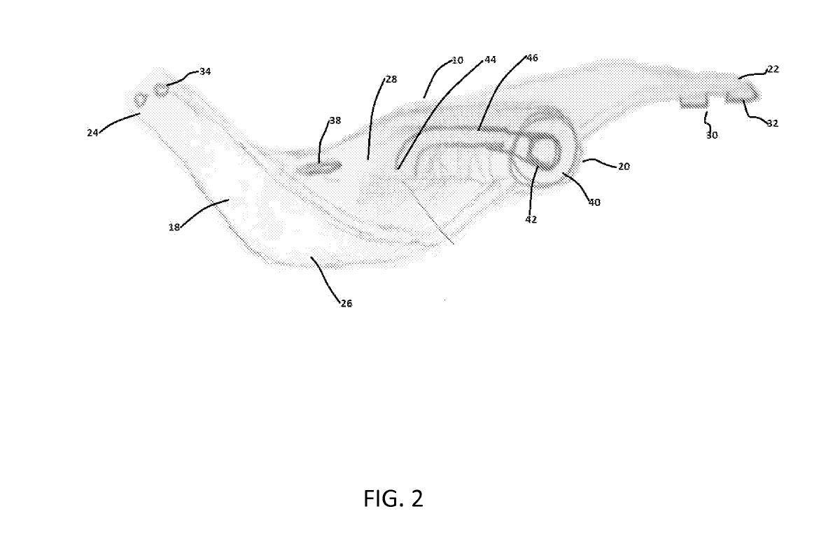

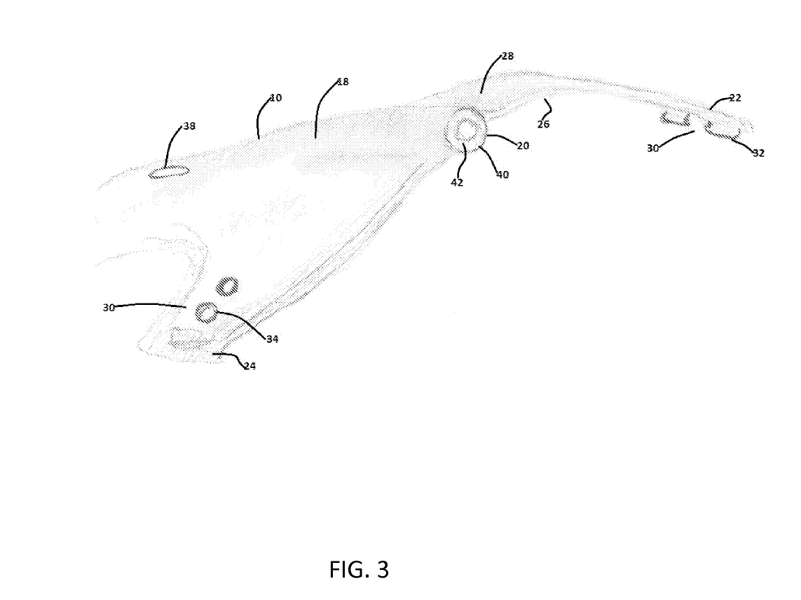

Embodiments of the present invention relate to an alignment device 10 used with a body part. FIG. 1 illustrates a foot 12 having heel 14 and insertion site proximate ankle 16. The alignment device 10 illustrated in FIGS. 2-3 includes a wrap 18 and an injection device 20 used for proper alignment and insertion of a metal device for treatment of medical conditions such as OAB and fecal incontinence.

In at least one embodiment the wrap 18 is a single unit having first end 22 and a second end 24. Wrap 18 further includes a first side 26 and a second side 28 opposite the first side 26, where the second side 28 is adapted to be placed against skin of the patient's foot 12. Wrap 10 further includes a securement device 30 for securing the first end 22 and second end 24. In the embodiment illustrated in FIGS. 2-3, securement device 30 includes one or more male snaps 32 located at first end 22 adapted to frictionally fit female snaps 34 located at second end 24.

In at least one embodiment, while snaps 32 and 34 are contemplated, other arrangements including hook and loops, hook and eyes, ties, snaps, buckles and the likes are contemplated. Furthermore, the wrap 10 is a single unit, although other embodiments such as two or more separate units removably secured together are contemplated as provide below.

FIGS. 2-3 depict one embodiment of the injection device 20. In the illustrated embodiment, injection device 20 comprises a tube 40 having a first end 42, a second end 44 and a bore 46 extending between first end 42 and a second end 44. As illustrated, second end 44 is adapted to be positioned at the insertion site proximate the ankle 16.

In one embodiment, the injection device 20 comprises a spring loaded tube, adapted to slowly twist and insert a metal device upon insertion. Additionally, the injection device 20 may comprise a guide tube used to insert the metal device by applying a mechanical or manual force to the guide tube and/or the metal device. Alternatively, the injection device may comprise a pneumatic device, a hydraulic device and the like and/or contain a plurality of metal devices, where the insertion depth of the metal devices is selectable.

In one or more embodiments of the present invention, the metal device is selected from a wire, a needle, an electrically conductive metal device and the like. Further the metal device is adapted to be electrically connectable to an electrical device, transmitting electrical pulses from the electrical device to the patient's foot.

In one or more embodiments, the wrap 10 may be a single unit made of a material selected from the group comprising an elastic gauze, elastic adhesive material, cotton, wool, nylon, and neoprene. In at least one embodiment, the wrap 10 defines a hole 38 which is adapted to receive the heel 14, such that the second end 44 is positioned proximate the ankle 16. Alternatively, the wrap 10 may be made up of two or more pieces which are made up of gel pads or air bladders, placed on opposite sides of the patient's ankle and secured in place by straps, belts, tape and the like.

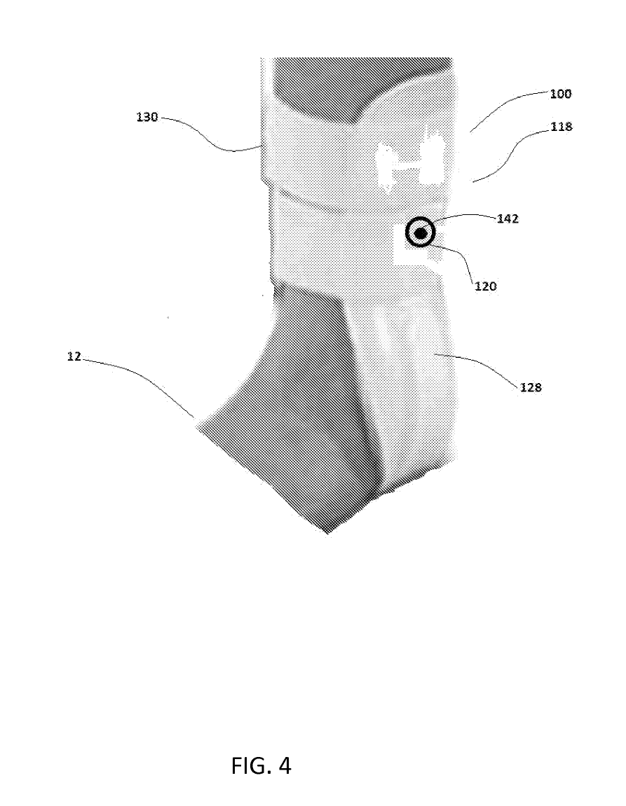

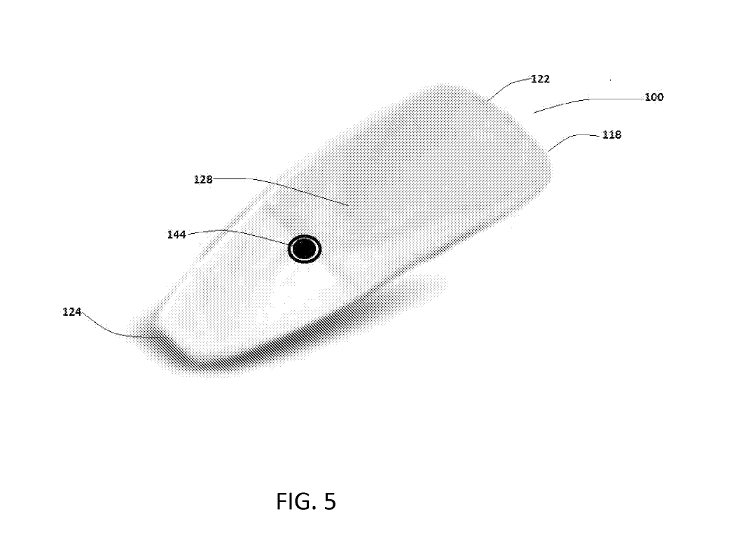

FIGS. 4-5 illustrate views of an alignment device 100 used with the ankle of FIG. 1 in accordance with another embodiment. In this embodiment the wrap 118 is a unit having first end 122 and a second end 124. Wrap 118 further includes a first side 126 and a second side 128 opposite the first side 126, where the second side 128 is adapted to be placed against skin of the patient's foot 12. Wrap 118 further includes a securement device 130 for securing the wrap 118. In the embodiment illustrated in FIGS. 4-5, securement device 130 includes one or more straps located proximate first end 122 adapted to couple to each other or the wrap 118 using a hook and loop arrangement for example.

FIGS. 4-5 depict one embodiment of the injection device 120. In the illustrated embodiment, injection device 120 comprises a tube having a first end 142, a second end 144 and a bore extending between first end 142 and a second end 144. As illustrated, second end 144 is adapted to be positioned at the insertion site proximate the ankle 16.

In one embodiment, the injection device 120 comprises a spring loaded tube, adapted to slowly twist and insert a metal device upon insertion. Additionally, the injection device 120 may comprise a guide tube used to insert the metal device by applying a mechanical or manual force to the guide tube and/or the metal device. Alternatively, the injection device 120 may comprise a pneumatic device, a hydraulic device and the like and/or contain a plurality of metal devices, where the insertion depth of the metal devices is selectable.

In one or more embodiments, the wrap 118 may be of a single unit of a material selected from a group comprising plastic, carbon fiber or any other suitable hard material while the securement device 130 may be of a material selected from the group comprising an elastic gauze, elastic adhesive material, cotton, wool, nylon, neoprene, ties, shoe strings, adjustable straps, rubber straps and the like. In at least one embodiment, the wrap 118 defines a hole which is adapted to receive the heel, such that the second end 144 is positioned proximate the ankle 16. Alternatively, the wrap 118 may be made up of two or more pieces which may include gel pads or air bladders, placed on opposite sides of the patient's ankle and secured in place by straps, belts, tape and the like.

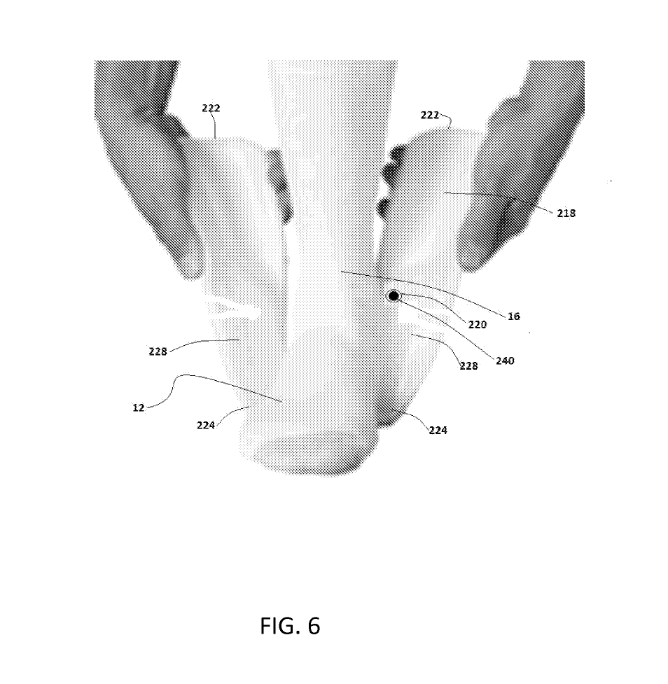

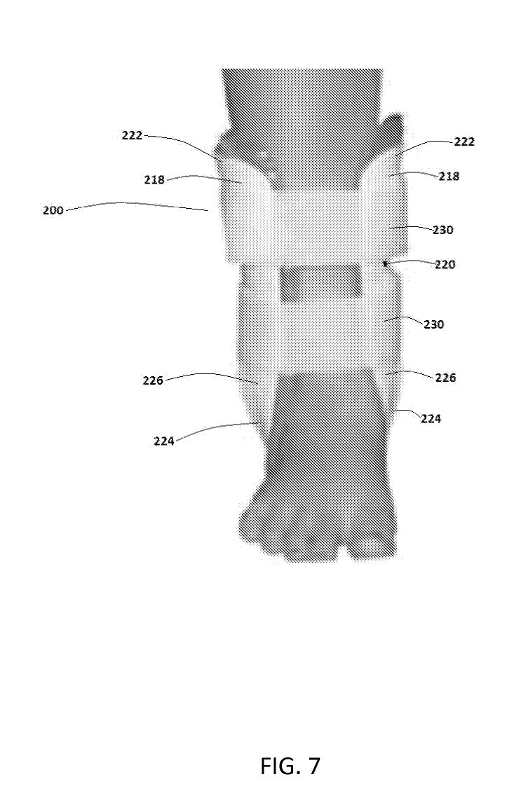

FIGS. 6-7 illustrate views of an alignment device 200 used with the ankle of FIG. 1 in accordance with another embodiment. In this embodiment the wrap 218 includes two units positionable on either side of angle 12, each unit of wrap 218 having first end 222 and a second end 224. Each unit of wrap 218 further includes a first side 226 and a second side 228 opposite the first side 226, where the second side 228 is adapted to be placed against skin of the patient's foot 12. Wrap 218 further includes a securement device 230 for securing the wrap 218. In the embodiment illustrated in FIGS. 6-7, securement device 230 includes one or more straps located between first end 222 and second end 224 adapted to couple to each other or the wrap 218 using a hook and loop arrangement although other means are contemplated.

FIGS. 6-7 depict one embodiment of the injection device 220 located on each of the units, positionable on both sides of the ankle 12. In the illustrated embodiment, each injection device 220 comprises a tube having a first end 242, a second end 244 and a bore extending between first end 242 and a second end 244. As illustrated, each second end 244 is adapted to be positioned at the insertion site proximate the ankle 16.

In one embodiment, the injection device 220 comprises a spring loaded tube, adapted to slowly twist and insert a metal device upon insertion. Additionally, the injection device 220 may comprise a guide tube used to insert the metal device by applying a mechanical or manual force to the guide tube and/or the metal device. Alternatively, the injection device 220 may comprise a pneumatic device, a hydraulic device and the like and/or contain a plurality of metal devices, where the insertion depth of the metal devices is selectable.

In one or more embodiments, the wrap 218 may of a material selected from a group comprising plastic, carbon fiber or any other suitable hard material while the securement device 230 may be of a material selected from the group comprising an elastic gauze, elastic adhesive material, cotton, wool, nylon, neoprene, ties, shoe strings, adjustable straps, rubber straps and the like.

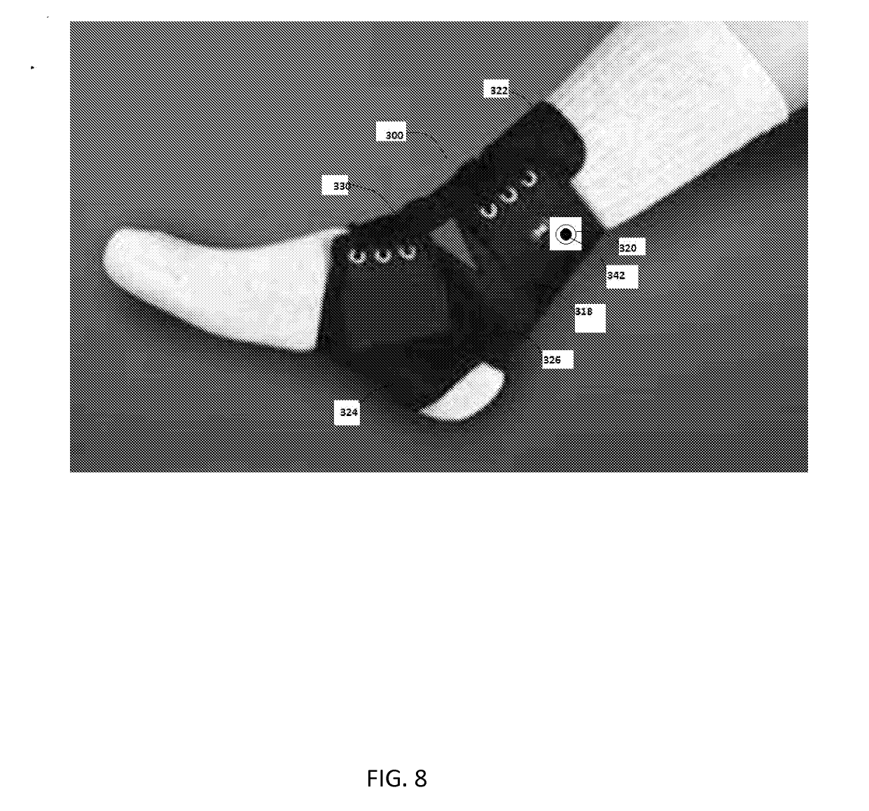

FIG. 8 illustrates a view of an alignment device 300 used with the ankle of FIG. 1 in accordance with another embodiment. In this embodiment the wrap 318 is a unit having first end 322 and a second end 324. Wrap 318 further includes a first side 326 and a second side opposite the first side 326, where the second side is adapted to be placed against skin of the patient's foot 12. Wrap 318 further includes a securement device 330 for securing the wrap 318. In the embodiment illustrated in FIG. 8, securement device 330 includes one or more ties or shoe strings extending from first end 322 towards second end 324 and is adapted to coupled or tied to each other.

FIG. 8 depicts one embodiment of the injection device 320. In the illustrated embodiment, injection device 320 comprises a tube having a first end 342, a second end and a bore extending between first end 342 and second end. As illustrated, the second end is adapted to be positioned at the insertion site proximate the ankle 16.

In one embodiment, the injection device 320 comprises a spring loaded tube, adapted to slowly twist and insert a metal device upon insertion. Additionally, the injection device 320 may comprise a guide tube used to insert the metal device by applying a mechanical or manual force to the guide tube and/or the metal device. Alternatively, the injection device 320 may comprise a pneumatic device, a hydraulic device and the like and/or contain a plurality of metal devices, where the insertion depth of the metal devices is selectable.

In one or more embodiments, the wrap 318 may be of a single unit of a material selected from a group comprising plastic, carbon fiber or any other suitable hard material while the securement device 330 may be of a material selected from the group comprising an elastic gauze, elastic adhesive material, cotton, wool, nylon, neoprene, ties, shoe strings, adjustable straps, rubber straps and the like. In at least one embodiment, the wrap 318 receives the heel, such that the second end is positioned proximate the ankle 16.

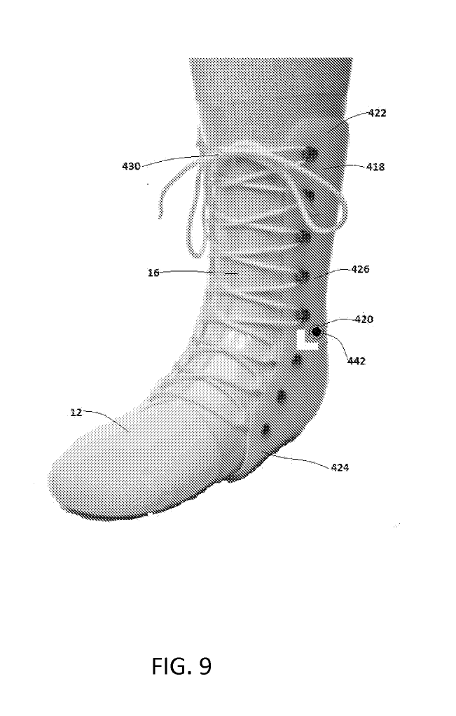

FIG. 9 illustrates a view of an alignment device 400 used with the ankle of FIG. 1 in accordance with another embodiment. In this embodiment the wrap 418 is a unit having first end 422 and a second end 424. Wrap 418 further includes a first side 426 and a second side opposite the first side 426, where the second side is adapted to be placed against skin of the patient's foot 12. Wrap 418 further includes a securement device 430 for securing the wrap 418. In the embodiment illustrated in FIG. 9, securement device 430 includes one or more straps ties or shoe strings extending from first end 422 towards second end 424 and is adapted to coupled or tied to each other.

FIG. 9 depicts one embodiment of the injection device 420. In the illustrated embodiment, injection device 420 comprises a tube having a first end 442, a second end and a bore extending between first end 442 and second end. As illustrated, the second end is adapted to be positioned at the insertion site proximate the ankle 16.

In one embodiment, the injection device 420 comprises a spring loaded tube, adapted to slowly twist and insert a metal device upon insertion. Additionally, the injection device 420 may comprise a guide tube used to insert the metal device by applying a mechanical or manual force to the guide tube and/or the metal device. Alternatively, the injection device 420 may comprise a pneumatic device, a hydraulic device and the like and/or contain a plurality of metal devices, where the insertion depth of the metal devices is selectable.

In one or more embodiments, the wrap 418 may be of a single unit of a material selected from a group comprising plastic, carbon fiber or any other suitable hard material while the securement device 430 may be of a material selected from the group comprising an elastic gauze, elastic adhesive material, cotton, wool, nylon, neoprene, ties, shoe strings, adjustable straps, rubber straps and the like. In at least one embodiment, the wrap 418 receives the heel, such that the second end is positioned proximate the ankle 16.

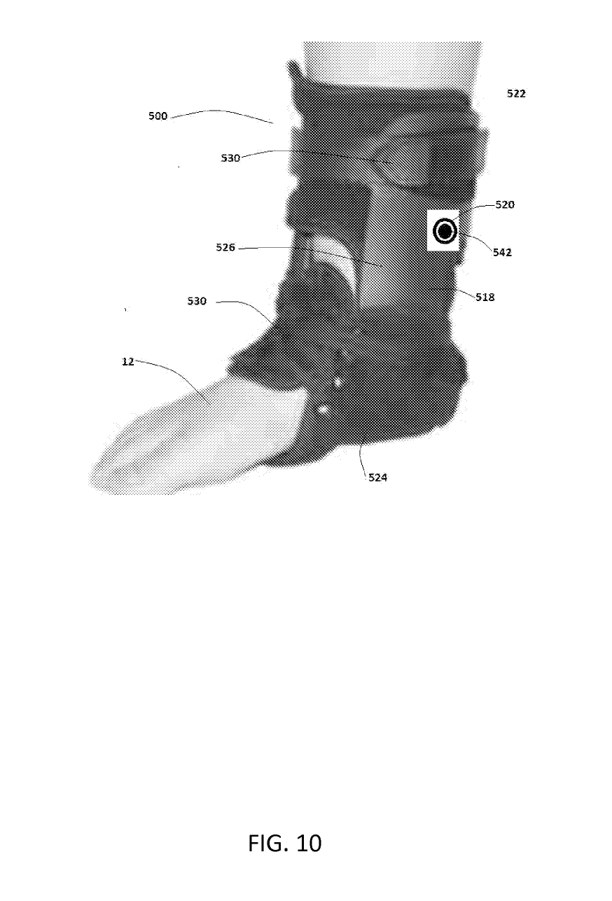

FIG. 10 illustrates a view of an alignment device 500 in accordance with another embodiment used with an ankle similar to that of FIG. 1. In this embodiment the wrap 518 is a unit having first end 522 and a second end 524. Wrap 518 further includes a first side 526 and a second side opposite the first side 526, where the second side is adapted to be placed against skin of the patient's foot 12. Wrap 518 further includes a securement device 530 for securing the wrap 518. In the embodiment illustrated in FIG. 10, securement device 530 includes one or more Velcro™ straps.

FIG. 10 depicts one embodiment of the injection device 520. In the illustrated embodiment, injection device 520 comprises a tube having a first end 542, a second end and a bore extending between first end 542 and second end. As illustrated, the second end is adapted to be positioned at the insertion site proximate the ankle 16.

In one embodiment, the injection device 520 comprises a spring loaded tube, adapted to slowly twist and insert a metal device upon insertion. Additionally, the injection device 520 may comprise a guide tube used to insert the metal device by applying a mechanical or manual force to the guide tube and/or the metal device. Alternatively, the injection device 520 may comprise a pneumatic device, a hydraulic device and the like and/or contain a plurality of metal devices, where the insertion depth of the metal devices is selectable.

In one or more embodiments, the wrap 518 may be of a single unit of a material selected from a group comprising plastic, carbon fiber or any other suitable hard material while the securement device 530 may be of a material selected from the group comprising an elastic gauze, elastic adhesive material, cotton, wool, nylon, neoprene, ties, shoe strings, adjustable straps, rubber straps and the like. In at least one embodiment, the wrap 518 receives the heel, such that the second end is positioned proximate the ankle 16.

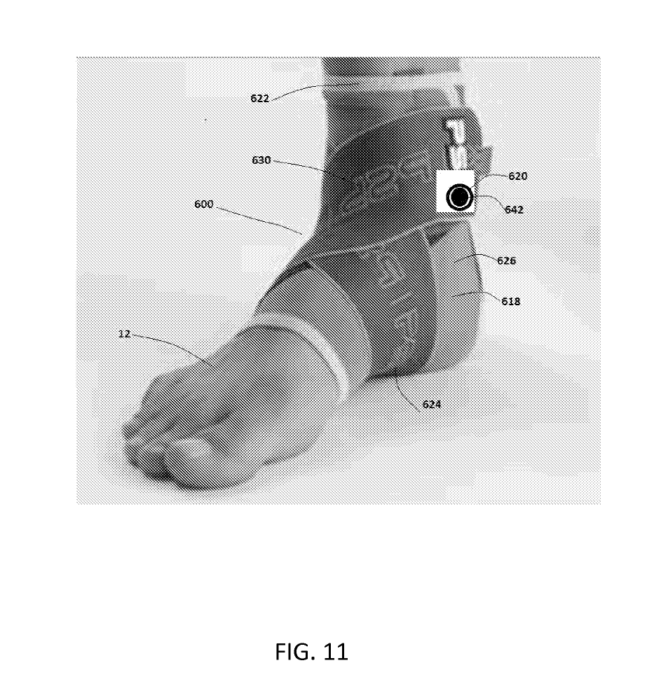

FIG. 11 illustrates a view of an alignment device 600 in accordance with another embodiment used with an ankle similar to that of FIG. 1. In this embodiment the wrap 618 is a unit having first end 622 and a second end 624. Wrap 618 further includes a first side 626 and a second side opposite the first side 626, where the second side is adapted to be placed against skin of the patient's foot 12. Wrap 618 further includes a securement device 630 for securing the wrap 618. In the embodiment illustrated in FIG. 11, securement device 630 includes one or more Velcro™ straps.

FIG. 11 depicts one embodiment of the injection device 620. In the illustrated embodiment, injection device 620 comprises a tube having a first end 642, a second end and a bore extending between first end 642 and second end. As illustrated, the second end is adapted to be positioned at the insertion site proximate the ankle 16.

In one embodiment, the injection device 620 comprises a spring loaded tube, adapted to slowly twist and insert a metal device upon insertion. Additionally, the injection device 620 may comprise a guide tube used to insert the metal device by applying a mechanical or manual force to the guide tube and/or the metal device. Alternatively, the injection device 620 may comprise a pneumatic device, a hydraulic device and the like and/or contain a plurality of metal devices, where the insertion depth of the metal devices is selectable.

In one or more embodiments, the wrap 618 may be of a single unit of a material selected from a group comprising plastic, carbon fiber or any other suitable hard material while the securement device 630 may be of a material selected from the group comprising an elastic gauze, elastic adhesive material, cotton, wool, nylon, neoprene, ties, shoe strings, adjustable straps, rubber straps and the like. In at least one embodiment, the wrap 618 receives the heel, such that the second end is positioned proximate the ankle 16.

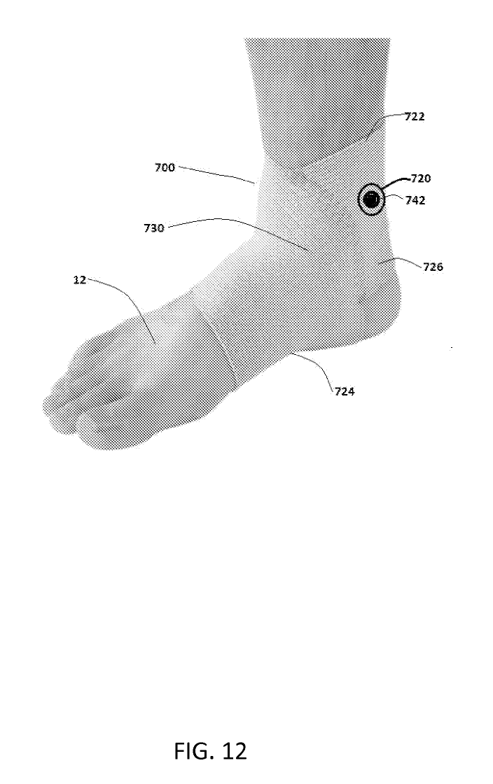

FIG. 12 illustrates a view of an alignment device 700 in accordance with another embodiment used with an ankle similar to that of FIG. 1. In this embodiment the wrap 718 is a unit having first end 722 and a second end 724. Wrap 718 further includes a first side 726 and a second side opposite the first side 726, where the second side is adapted to be placed against skin of the patient's foot 12. Wrap 718 further includes a securement device 730 for securing the wrap 718. In the embodiment illustrated in FIG. 12, securement device 730 includes one or more Velcro™ straps.

FIG. 12 depicts one embodiment of the injection device 720. In the illustrated embodiment, injection device 720 comprises a tube having a first end 742, a second end and a bore extending between first end 742 and second end. As illustrated, the second end is adapted to be positioned at the insertion site proximate the ankle 16.

In one embodiment, the injection device 720 comprises a spring loaded tube, adapted to slowly twist and insert a metal device upon insertion. Additionally, the injection device 720 may comprise a guide tube used to insert the metal device by applying a mechanical or manual force to the guide tube and/or the metal device. Alternatively, the injection device 720 may comprise a pneumatic device, a hydraulic device and the like and/or contain a plurality of metal devices, where the insertion depth of the metal devices is selectable.

In one or more embodiments, the wrap 718 may be of a single unit of a material selected from a group comprising plastic, carbon fiber or any other suitable hard material while the securement device 730 may be of a material selected from the group comprising an elastic gauze, elastic adhesive material, cotton, wool, nylon, neoprene, ties, shoe strings, adjustable straps, rubber straps and the like. In at least one embodiment, the wrap 718 receives the heel, such that the second end is positioned proximate the ankle 16.

FIG. 2-3 illustrates that the injection device 20 is positioned such that the metal device is inserted into ankle 12 at about a 45 degree angle to a plane defined by ankle 12. However embodiments are contemplated in which the metal device is inserted at different angles, for example, in the embodiments illustrated in FIGS. 4-12, the injection devices 120/220/320 and 420 are positioned such that the metal device is inserted at about a 90 degree angle to a plane defined by ankle 12. However, that angle could range from about 15 degrees to about 165 degrees to the plane defined by ankle 12 or more specifically between about 30 degrees and 90 degrees to the plane defined by ankle 12.

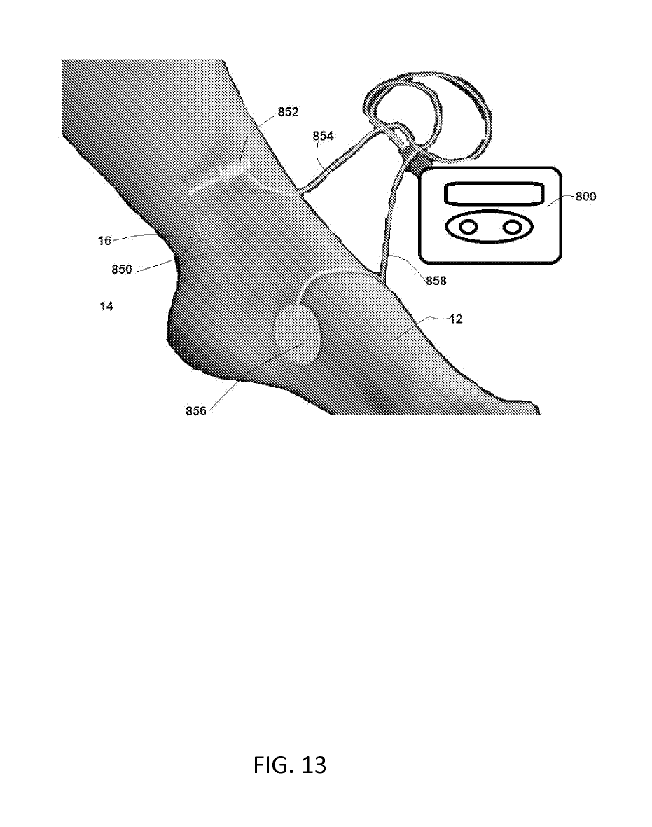

FIG. 13 illustrates an example of a TENS device 800 used with foot 12 having heel 14 and insertion site proximate ankle 16. In the illustrated embodiment, TENS device 800 is coupled to a metal device or needle 850 via connector or clip 852 and cable 854, where the metal device or needle is inserted a couple of inches into the area proximate or above the ankle 16. FIG. 13 further illustrates a patch or electrode 856 attached to the bottom of foot 12 via cable 858. TENS device 800 creates electrical pulses transmitted to the patient's nerves and treat OAB and FI.

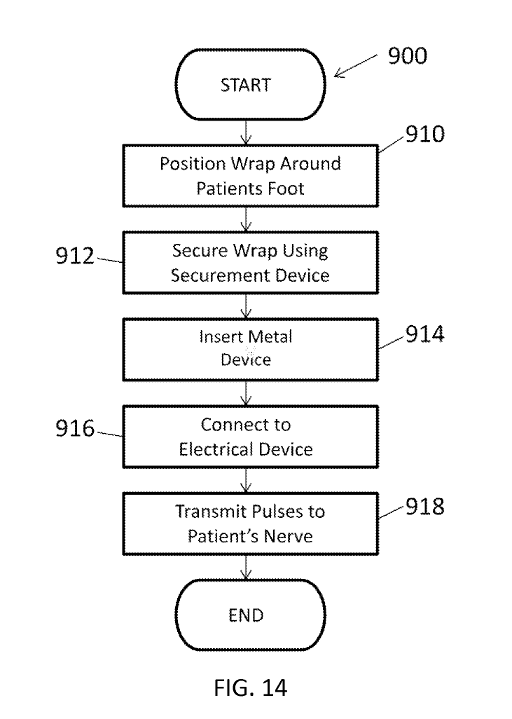

FIG. 14 illustrates one or more embodiments relating to a method generally designate 900, for inserting a metal device into a portion of a human body (an ankle for example) using any of the alignment devices as provided above. The method 900 comprises positioning the wrap around the patient's foot 12 and the second side is adapted to be placed against skin of the patient's ankle 12 and injection device 20 is positioned adjacent ankle, block 910.

The alignment device 10 is then secured using the securement device, block 912. Method 900 further comprises slowly twisting and inserting the metal device into the patient, block 914. The metal device is connected to an electrical device, block 916. Electrical pulses are transmitted to the patient's nerves from the electrical device and the metal device, block 918. While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. An alignment device, comprising:
a wrap;
an injection device for proper alignment and insertion of a metal device.

2. The alignment device of claim 1, wherein the wrap is a single sleeve unit.

3. The alignment device of claim 1 wherein the wrap has a first end, a second end and a securement device for securing the first end to the second end.

4. The alignment device of claim 3 wherein the securement device is selected from the group comprising hook and loops, hook and eyes, ties, snaps, buckles and buttons.

5. The alignment device of claim 1 wherein the injection device comprises a spring loaded tube.

6. The alignment device of claim 5 wherein the spring loaded tube slowly twists and inserts the metal device upon insertion.

7. The alignment device of claim 1 wherein the injection device comprises a guide tube used to insert the metal device by applying a mechanical force to the guide tube.

8. The alignment device of claim 1 wherein the metal device is electrically connectable to an electrical device.

9. The alignment device of claim 1 wherein the metal device is selected from the group composing a wire, a needle and an electrically conductive metal device.

10. The alignment device of claim 1 wherein the material for the wrap is selected from the group comprising an elastic gauze, elastic adhesive material, cotton, wool, nylon and neoprene.

11. An alignment device for use with a body for treatment of medical conditions such as OAB and fecal incontinence, comprising:
a wrap having a first end and a second end for use in contact with a portion of the body;
a securement device for securing the first end to the second end; and
an alignment device defining a tube having a first open end in contact with the portion of the human body and at a predetermined angle thereto, the alignment device providing proper alignment and insertion of a metal device.

12. The alignment device of claim 11 wherein the tube is a spring loaded tube that slowly inserts and twists the metal device upon insertion.

13. The alignment device of claim 11 wherein the predetermined angle is an angle between 15 degrees and 165 degrees to an angle defined by the portion of the body.

14. The alignment device of claim 11 wherein the predetermined angle is between about 30 degrees and about 90 degrees to an angle defined by the portion of the body.

15. The alignment device of claim 11 wherein the metal device is electrically connectable to an electrical device.

16. The alignment device of claim 11 wherein the injection device comprises a guide tube used to insert the metal device by applying a mechanical force to the guide tube.

17. The alignment device of claim 11 wherein the metal device is selected from the group composing a wire, a needle and an electrically conductive metal device.

18. The alignment device of claim 11 wherein the material for the wrap is selected from the group comprising an elastic gauze, elastic adhesive material, cotton, wool, nylon and neoprene.

19. The alignment device of claim 11 wherein the securement device is selected from the group comprising hook and loops, hook and eyes, ties, snaps, buckles and buttons.

20. A method for inserting a metal device into a portion of a human body using an alignment device, the alignment device comprising:
a wrap; and
an injection device for proper alignment and insertion of the metal device;
the method comprising:
positioning the wrap on the portion of the human body; and
slowly twisting and inserting the metal device.
